(19) United States
(12) Patent Application Publication
(10) Pub. No.: US 2024/0335338 A1
(43) Pub. Date: Oct. 10, 2024

(54) Attachable Animal Medical Kit Device

(71) Applicant: Paula Foster, Millsap, TX (US)

(72) Inventor: Paula Foster, Millsap, TX (US)

(21) Appl. No.: 18/616,288

(22) Filed: Mar. 26, 2024

Related U.S. Application Data
(60) Provisional application No. 63/457,471, filed on Apr. 6, 2023.

(51) Int. Cl. A61F 17/00

(52) U.S. Cl. CPC A61F 17/00

(57) ABSTRACT

An animal medical kit device is disclosed that comprises a body component that is configured in a cylindrical or rectangular shape with a fastener means for securing the body component to a pet's leash, collar, harness, etc. The body component can be used to store a syringe, nasal spray, an insulin needle, various pills, or other medications related to the pet. Further, the device comprises a scannable QR code secured on a tag which can be secured to the pet's collar. The QR code discloses detailed medication-related directions and dosages once scanned.

### CROSS-REFERENCE TO RELATED APPLICATION

The present application claims priority to, and the benefit of, U.S. Provisional Application No. 63/457,471, which was filed on Apr. 6, 2023, and is incorporated herein by reference in its entirety.

### FIELD OF THE INVENTION

The present invention relates generally to the field of animal medical kit devices. More specifically, the present invention relates to a device that ensures a pet always has access to daily medication. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in animal medical kit devices. Generally, many pets are prescribed medicine that they must take daily. However, pets may become lost. If the pet is found by an individual who is not the pet's owner, said individual may be unaware the pet needs any medication and further does not possess said medication. As a result, the pet may suffer adverse effects without the medication.

Further, lost pets which are found may not be able to be returned for some time, if the owners are at work and/or away from their homes for an extended period of time, and may need their medication given before the owner returns. Thus, there's a need for a means to alert someone who finds a pet that the pet requires medication, but also where the medication is and how much to give the pet.

Accordingly, there is a demand for an improved animal medical kit device that can ensure a pet always has access to daily medication. More particularly, there is a demand for an animal medical kit device that comprises a scannable QR code to disclose detailed medication-related directions and dosages.

Therefore, there exists a long-felt need in the art for an animal medical kit device that provides users with a means for ensuring a pet always has access to daily medication. There is also a long-felt need in the art for an animal medical kit device that can be used to store medicine on a pet at all times. Further, there is a long-felt need in the art for an animal medical kit device that can be used to store medicine on a pet at all times on the collar, harness, or leash of the pet. Moreover, there is a long-felt need in the art for a device that comprises a waterproof, fabric case with a fastener for securing to the animal's collar, harness, or leash. Further, there is a long-felt need in the art for an animal medical kit device that also comprises a pill container with a child-proof lid for storing pills or other medications. Finally, there is a long-felt need in the art for an animal medical kit device that comprises a QR code for providing detailed medication-related directions and dosages.

The subject matter disclosed and claimed herein, in one embodiment thereof, comprises an animal medical kit device. The device is an attachable animal medical kit device. The animal medical kit device comprises a body component that is configured in a cylindrical or rectangular shape. If cylindrical, the body component is comprised of a waterproof, fabric case. If rectangular, the body component is comprised of an insulated, plastic container. Both configurations would comprise a child-proof lid and a fastener means for securing the body component to a pet's leash, collar, harness, etc. In one embodiment, the fastener means is a pair of snapping loops which secure around the ends of the body component then snap onto the collar for use. In another embodiment, the fastener means is an adhesive-backed case which comprises a button snap lid. The adhesive-backed case is secured directly onto the leash or harness, as desired. In yet another embodiment, the fastening means is a keychain or carabiner that secures to the body component and then to the leash for use. The body component can be used to store a syringe, nasal spray, an insulin needle, various pills, or other medications related to the pet. Further, the device comprises a scannable QR code secured on a tag which can be secured to the pet's collar. The QR code discloses detailed medication-related directions and dosages once scanned.

In this manner, the animal medical kit device of the present invention accomplishes all of the foregoing objectives and provides users with a device that ensures a pet always has access to daily medication. The device is a container that secures to a pet's leash, collar, harness, etc. The device includes a QR code for medication-related directions.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an animal medical kit device. The device is an attachable animal medical kit device. The animal medical kit device comprises a body component that is configured in a cylindrical or rectangular shape. Both configurations would comprise a child-proof lid and a fastener means for securing the body component to a pet's leash, collar, harness, etc. The body component can be used to store a syringe, nasal spray, an insulin needle, various pills, or other medications related to the pet. Further, the device comprises a scannable QR code secured on a tag which can be secured to the pet's collar. The QR code discloses detailed medication-related directions and dosages once scanned.

In one embodiment, an animal medical kit device is utilized to ensure that a pet always has access to daily medication, even if the pet becomes lost and/or separated from their owner for an extended period of time. The device not only provides access to the daily medication but also includes a QR code that can be scanned to access detailed medication-related directions and dosages pertaining to the pet medication.

In one embodiment, the animal medical kit device comprises a body component that is configured to retain pet medications, such as, but not limited to, a syringe, nasal spray, an insulin needle, various pills of different shapes, sizes, and quantities, or any other suitable medications related to the pet, as is known in the art. The body component can be configured in any suitable shape and size as is known in the art, depending on the needs and/or wants of a user. However, the body component typically has two main configurations, one being a cylindrical configuration for retaining a syringe, nasal spray, an insulin needle, as well as a variety of pills, etc. Typically, the cylindrical configuration is a thermos-style, insulated container to keep medications cold, if needed. Specifically, the thermos-style, insulated container can be stored in the freezer with the required medications and then secured to the pet, as needed, to keep the medications cold all day long. Further, the lid component of the thermos-style, insulated container comprises a twist-on/twist-off, child-proof locking lid to secure the medication within the container and prevent small children from accessing the medication. The body component of the cylindrical configuration comprises a bottom face, a cylindrical sidewall, extending vertically upwards, and an open top. The child-proof locking lid secures and closes the open top, retaining the contents within the body component. Typically, the cylindrical configuration is a plastic container available in different sizes.

In one embodiment, a second configuration for the body component includes a rectangular configuration for retaining a plurality of pills or other medication. The rectangular configuration can be manufactured in multiple sizes, with a small size retaining up to six large pills, the medium size retaining additional pills, and the large size retaining the most amount of pills of all sizes available. The lid component of the rectangular configuration comprises a hinged lid. Typically, the rectangular configuration is a plastic container of various sizes. The body component for the rectangular configuration comprises a bottom face, an open top, opposing side walls, and opposing front and back walls. The hinges of the hinged lid are secured to a top edge of the back wall to allow the hinged lid to pivot down and close the open top, retaining the contents within the body component.

In one embodiment, the body component is secured to a pet's leash, collar, harness, etc., or any other suitable pet retaining device as is known in the art. Specifically, the body component is secured via a fastener means. Fastener means can include any suitable securing means as is known in the art, such as silicone snapping loops, an adhesive housing, Velcro, snaps, etc., which allows the body component to be secured to a collar, leash, harness, etc.

In one embodiment, when the body component is secured to a collar, the fastener means typically comprises silicone snapping loops. For example, if the body component is a cylindrical configuration, a pair of silicone snapping loops secure to each end of the body component. Specifically, the snapping loops each wrap completely around an end of the body component and secure together, a C-shaped component of the snapping loops then engage the collar, securing the body component in place. Once, the C-shaped component are secured around the width of the collar, the ends are snapped into place, preventing the body component from being removed. Accordingly, the body component is attached to the collar at two spaced apart points by the snapping loops at either end of the body component, so that the body component will not swing about annoyingly during a walk. If the body component is a rectangular configuration, the pair of silicone snapping loops are secured to a covering with a lid flap, which is sized and shaped to retain the rectangular body component within. The lid flap then secures to the covering to retain the body component, during use. The silicone snapping loops would be positioned on either side of the body component covering and secure around the collar, as stated supra. Generally, the silicone snapping loops can secure the body component to a pet's collar or harness.

In one embodiment, when the body component is secured to a harness, the fastener means typically comprises an adhesive or Velcro-backed fabric case with a lid flap. For example, if the body component is a cylindrical configuration, the fabric case is sized and shaped to retain the body component within. The lid flap then secures to the fabric case to retain the body component, during use. The fabric case is typically a waterproof, fabric case that comprises a back side of adhesive or Velcro, or any other suitable securing means. Once the body component is in place within the fabric case, the fabric case is then adhered to the harness or leash, as needed. Typically, the body component is secured to the harness, near the pet's back but can be secured in any suitable position, as is known in the art. Generally, the exterior of the fabric case can be formed from nylon or any other type of weather-resistant and/or water-resistant fabrics.

In one embodiment, when the body component is secured to a leash, the fastener mean typically comprises a keychain or carabiner. In this embodiment, the keychain or carabiner is secured to an end of the body component via any suitable securing means. The keychain or carabiner is then releasably secured to the handle or any other suitable position on the leash via the typical attachment of a keychain or carabiner.

In one embodiment, the device comprises a scannable QR code. The scannable QR code would be printed on a tag that is secured to the pet's collar or other suitable device. The tag can be printed silicone or any other suitable material as is known in the art. Each tag would be printed with a customized QR code. Specifically, the QR code would be customized for each individual pet. The QR code would contain pet information as well as medication information. Thus, once a user scans the QR code, pet information such as residence and owner phone numbers are displayed, as well as medical information, such as detailed medication-related directions and dosages specific to the pet and its medications.

In one embodiment, a plurality of reflective strips can be applied to an exterior surface of the body component. The purpose of these reflective strips would be to provide additional safety for the dog or other pet as well as the pet's owner. The reflective strips can be affixed to the exterior of the body component in virtually any manner such as reflective paint or can be removable from the body component through the use of Velcro®-type strips.

In one embodiment, the animal medical kit device can be used by the pet to transport human medicine. For example, a service dog could carry medicine for its handler with the disclosed animal medical kit.

In yet another embodiment, the animal medical kit device comprises a plurality of indicia.

In yet another embodiment, a method of ensuring a pet always has access to daily medication is disclosed. The method includes the steps of providing an animal medical kit device comprising a body component and lid component for storing pet medications. The method also comprises inserting the pet medications into the body component and securing the lid component. Further, the method comprises securing the body component to a pet's collar, leash, or harness. Finally, the method comprises providing a scannable QR code on a pet's collar for disclosing detailed medication-related directions and dosages once scanned.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the animal medical kit device of the present invention showing the device in accordance with the disclosed architecture;

FIGS. 2A-E illustrate a perspective view of one embodiment of the animal medical kit device of the present invention showing the various configurations of body components in accordance with the disclosed architecture;

FIGS. 3A-D illustrate a perspective view of one embodiment of the animal medical kit device of the present invention showing the different fastening means in accordance with the disclosed architecture;

FIG. 4 illustrates a perspective view of one embodiment of the animal medical kit device of the present invention showing the device secured to a pet's collar in accordance with the disclosed architecture;

FIG. 5 illustrates a perspective view of one embodiment of the animal medical kit device of the present invention showing the device secured to a pet's leash and harness in accordance with the disclosed architecture;

FIG. 6 illustrates a perspective view of one embodiment of the animal medical kit device of the present invention showing the device secured to a pet's harness in accordance with the disclosed architecture;

FIGS. 7A-K illustrate a perspective view of one embodiment of the animal medical kit device of the present invention showing different embodiments of the device in accordance with the disclosed architecture; and

FIG. 8 illustrates a flowchart showing the method of ensuring a pet always has access to daily medication in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an animal medical kit device that provides users with a means for ensuring a pet always has access to daily medication. There is also a long-felt need in the art for an animal medical kit device that can be used to store medicine on a pet at all times. Further, there is a long-felt need in the art for an animal medical kit device that can be used to store medicine on a pet at all times on the collar, harness, or leash of the pet. Moreover, there is a long-felt need in the art for a device that comprises a waterproof, fabric case with a fastener for securing to the animal's collar, harness, or leash. Further, there is a long-felt need in the art for an animal medical kit device that also comprises a pill container with a child-proof lid for storing pills or other medications. Finally, there is a long-felt need in the art for an animal medical kit device that comprises a QR code for providing detailed medication-related directions and dosages.

The present invention, in one exemplary embodiment, is a novel animal medical kit device. The device comprises a body component that is configured in a cylindrical or rectangular shape. Both configurations would comprise a child-proof lid and a fastener means for securing the body component to a pet's leash, collar, harness, etc. The body component can be used to store a syringe, nasal spray, an insulin needle, various pills, or other medications related to the pet. Further, the device comprises a scannable QR code secured on a tag which can be secured to the pet's collar. The QR code discloses detailed medication-related directions and dosages once scanned. The present invention also includes a novel method of ensuring a pet always has access to daily medication. The method includes the steps of providing an animal medical kit device comprising a body component and lid component for storing pet medications. The method also comprises inserting the pet medications into the body component and securing the lid component. Further, the method comprises securing the body component to a pet's collar, leash, or harness. Finally, the method comprises providing a scannable QR code on a pet's collar for disclosing detailed medication-related directions and dosages once scanned.

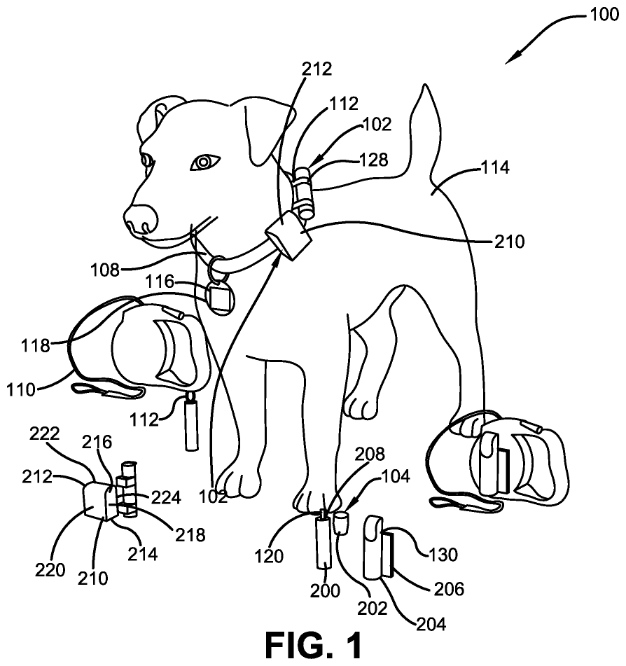

Referring initially to the drawings, FIG. 1 illustrates a perspective view of one embodiment of the animal medical kit device 100 of the present invention. In the present embodiment, the animal medical kit device 100 is an improved animal medical kit device 100 that provides a user with a device for ensuring a pet 114 always has access to daily medication. Specifically, the animal medical kit device 100 comprises a body component 102 with a lid component 104 that is secured to a pet's harness 106, collar 108, leash 110, etc., via a fastener means 112. The body component 102 is used to store a syringe 120, nasal spray 122, an insulin needle 124, various pills 126, or other medications related to the pet 114. Further, the device 100 comprises a scannable QR code 116 secured on a tag 118 which can be secured to the pet's collar 108. The QR code 116 discloses detailed medication-related directions and dosages once scanned.

Generally, the animal medical kit device 100 is utilized to ensure that a pet 114 always has access to daily medication, even if the pet 114 becomes lost and/or separated from their owner for an extended period of time. The device 100 not only provides access to the daily medication but also includes a QR code 116 that can be scanned to access detailed medication-related directions and dosages pertaining to the pet medication.

Furthermore, the body component 102 is secured to a pet's leash 110, collar 108, harness 106, etc., or any other suitable pet retaining device as is known in the art. Specifically, the body component 102 is secured via a fastener means 112. Fastener means 112 can include any suitable securing means as is known in the art, such as silicone snapping loops 128, an adhesive housing 130, Velcro, snaps, etc., which allows the body component 102 to be secured to a collar 108, leash 110, harness 106, etc.

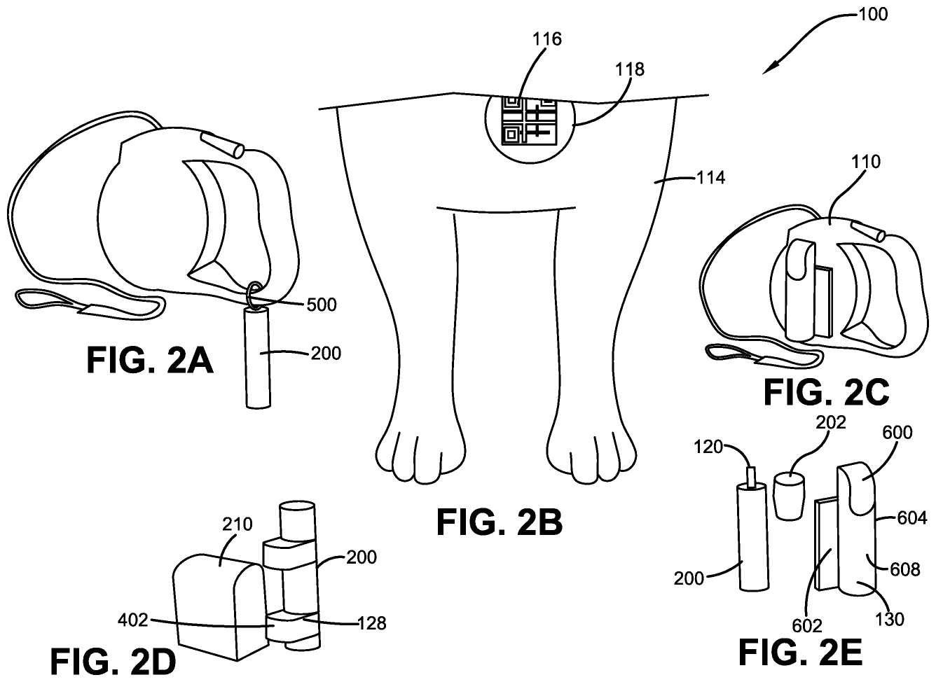

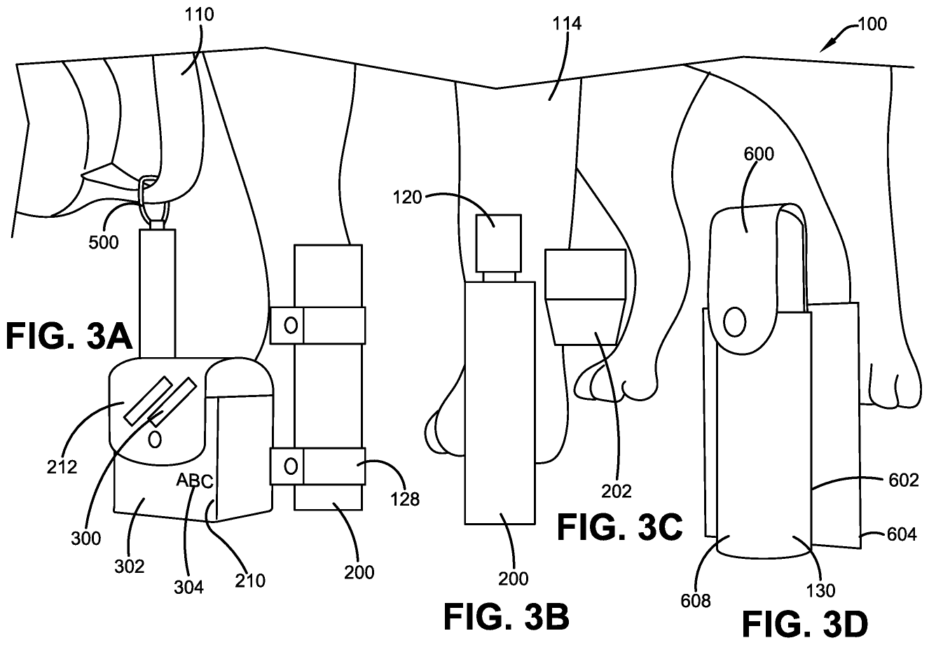

As shown in FIGS. 2-3, the animal medical kit device 100 comprises a body component 102 that is configured to retain pet medications, such as, but not limited to, a syringe 120, nasal spray 122, an insulin needle 124, various pills 126 of different shapes, sizes, and quantities, or any other suitable medications related to the pet 114, as is known in the art. The body component 102 can be configured in any suitable shape and size as is known in the art, depending on the needs and/or wants of a user. However, the body component 102 typically has two main configurations, one being a cylindrical configuration 200 for retaining a syringe 120, nasal spray 122, an insulin needle 124, as well as a variety of pills 126, etc. Typically, the cylindrical configuration 200 is a thermos-style, insulated container to keep medications cold, if needed. Specifically, the thermos-style, insulated container 200 can be stored in the freezer with the required medications and then secured to the pet 114, as needed, to keep the medications cold all day long. Further, the lid component 104 of the thermos-style, insulated container 200 comprises a twist-on/twist-off, child-proof locking lid 202 to secure the medication within the container 200 and prevent small children from accessing the medication. The body component 102 of the cylindrical configuration 200 comprises a bottom face 204, a cylindrical sidewall 206, extending vertically upwards, and an open top 208. The child-proof locking lid 202 secures and closes the open top 208, retaining the contents within the body component 102. Typically, the cylindrical configuration 200 is a plastic container available in different sizes.

Furthermore, a second configuration for the body component 102 includes a rectangular configuration 210 for retaining a plurality of pills 126 or other medication. The rectangular configuration 210 can be manufactured in multiple sizes, with a small size retaining up to six large pills 126, the medium size retaining additional pills 126, and the large size retaining the most amount of pills 126 of all sizes available. The lid component 104 of the rectangular configuration 210 comprises a hinged lid 212. Typically, the rectangular configuration 210 is a plastic container of various sizes. The body component 102 for the rectangular configuration 210 comprises a bottom face 214, an open top 216, opposing side walls 218, and opposing front and back walls 220. The hinges 222 of the hinged lid 212 are secured to a top edge 224 of the back wall 220 to allow the hinged lid 212 to pivot down and close the open top 216, retaining the contents within the body component 102.

Additionally, the device 100 comprises a scannable QR code 116. The scannable QR code 116 would be printed on a tag 118 that is secured to the pet's collar 108 or other suitable device. The tag 118 can be printed silicone or any other suitable material as is known in the art. Each tag 118 would be printed with a customized QR code 116. Specifically, the QR code 116 would be customized for each individual pet 114. The QR code 116 would contain pet information as well as medication information. Thus, once a user scans the QR code 116, pet information such as residence and owner phone numbers are displayed, as well as medical information, such as detailed medication-related directions and dosages specific to the pet 114 and its medications.

In one embodiment, a plurality of reflective strips 300 can be applied to an exterior surface 302 of the body component 102. The purpose of these reflective strips 300 would be to provide additional safety for the dog or other pet 114 as well as the pet's owner. The reflective strips 300 can be affixed to the exterior 302 of the body component 102 in virtually any manner such as reflective paint or can be removable from the body component 102 through the use of Velcro®-type strips.

In one embodiment, the animal medical kit device 100 can be used by the pet 114 to transport human medicine. For example, a service dog 114 could carry medicine for its handler with the disclosed animal medical kit device 100.

In yet another embodiment, the animal medical kit device 100 comprises a plurality of indicia 304. The body component 102 of the device 100 may include advertising, a trademark, or other letters, designs, or characters, printed, painted, stamped, or integrated into the body component 102, or any other indicia 304 as is known in the art. Specifically, any suitable indicia 304 as is known in the art can be included, such as, but not limited to, patterns, logos, emblems, images, symbols, designs, letters, words, characters, animals, advertisements, brands, etc., that may or may not be pet, medication, or brand related.

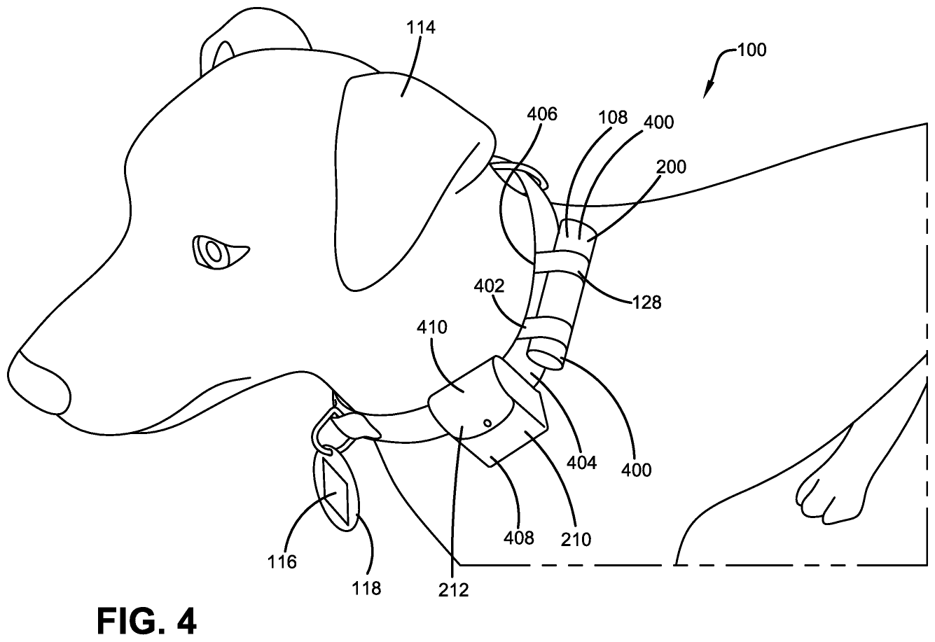

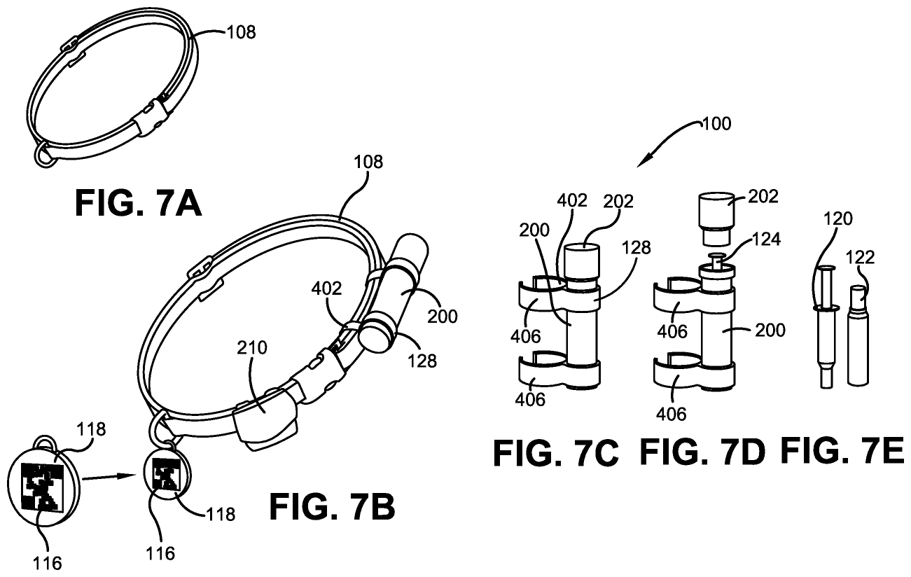

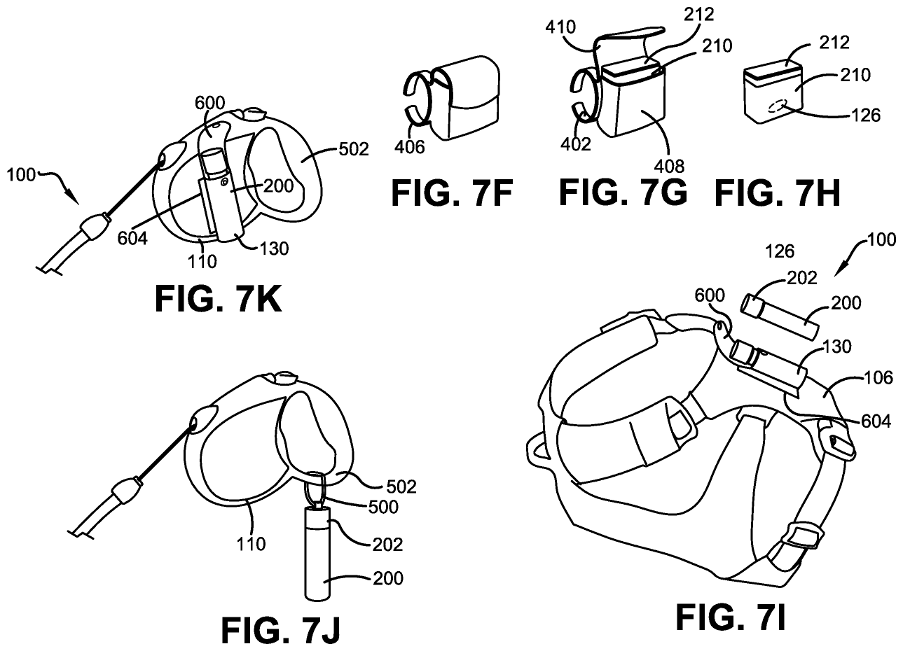

As shown in FIGS. 4 and 7A-H, when the body component 102 is secured to a collar 108, the fastener means 112 typically comprises silicone snapping loops 128. For example, if the body component 102 is a cylindrical configuration 200, a pair of silicone snapping loops 128 secure to each end 400 of the body component 102. Specifically, the snapping loops 128 each wrap completely around an end 400 of the body component 102 and secure together, a C-shaped component 402 of the snapping loops 128 then engage the collar 108, securing the body component 102 in place. Once, the C-shaped component 402 are secured around the width 404 of the collar 108, the ends 406 are snapped into place, preventing the body component 102 from being removed. Accordingly, the body component 102 is attached to the collar 108 at two spaced apart points by the snapping loops 128 at either end 400 of the body component 102, so that the body component 102 will not swing about annoyingly during a walk. If the body component 102 is a rectangular configuration 210, the pair of silicone snapping loops 128 are secured to a covering 408 with a lid flap 410, which is sized and shaped to retain the rectangular body component 210 within. The lid flap 410 then secures to the covering 408 to retain the body component 102, during use. The silicone snapping loops 128 would be positioned on either end 400 of the body component 102 covering 408 and secured around the collar 108, as stated supra. Generally, the silicone snapping loops 128 can secure the body component 102 to a pet's collar 108 or harness 106.

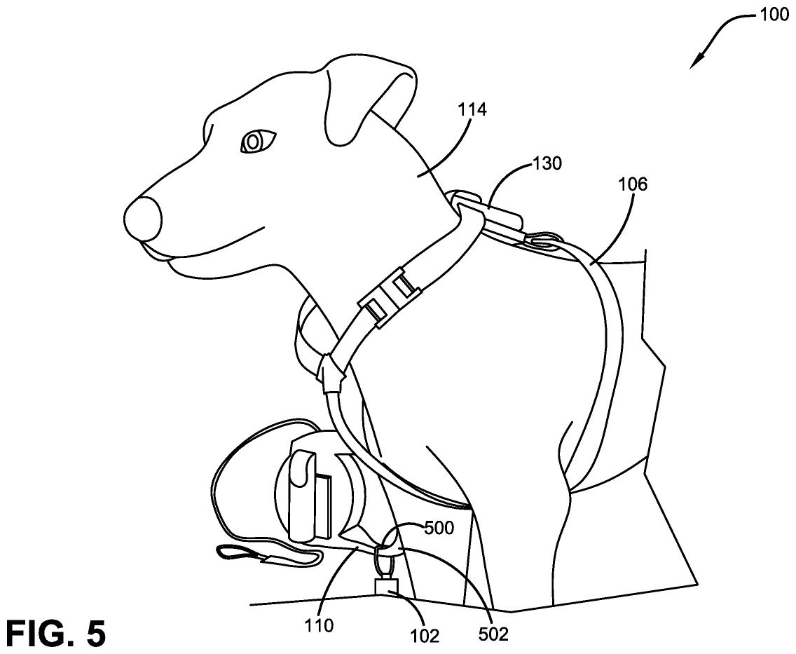

As shown in FIGS. 5 and 7J-K, when the body component 102 is secured to a leash 110, the fastener means 112 typically comprises a keychain or carabiner 500. In this embodiment, the keychain or carabiner 500 is secured to an end 400 of the body component 102 via any suitable securing means. The keychain or carabiner 500 is then releasably secured to the handle 502 or any other suitable position on the leash 110 via the typical attachment of a keychain or carabiner 500.

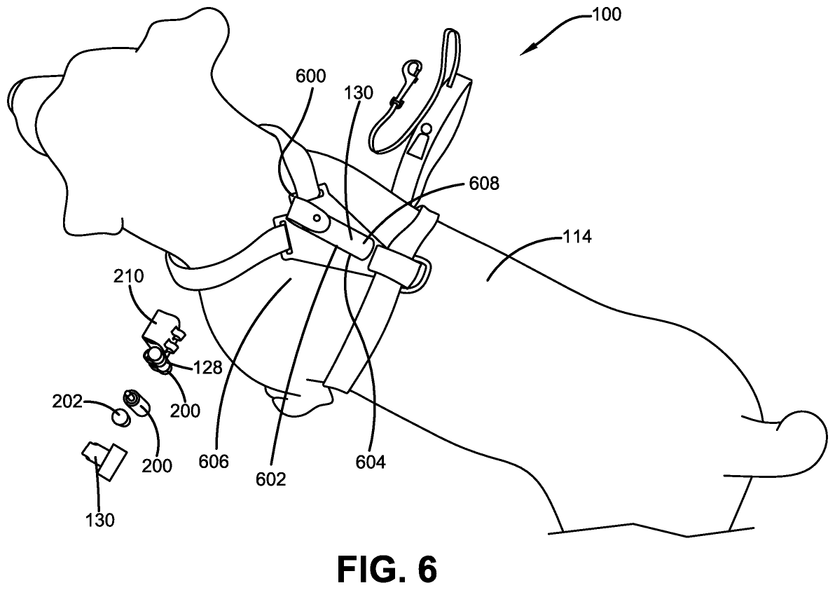

As shown in FIGS. 6 and 71, when the body component 102 is secured to a harness 106, the fastener means 112 typically comprises an adhesive or Velcro-backed fabric case 130 with a lid flap 600. For example, if the body component 102 is a cylindrical configuration 200, the fabric case 130 is sized and shaped to retain the body component 102 within. The lid flap 600 then secures to the fabric case 130 to retain the body component 102, during use. The fabric case 130 is typically a waterproof, fabric case 130 that comprises a back side 602 of adhesive 604 or Velcro, or any other suitable securing means. Once the body component 102 is in place within the fabric case 130, the fabric case 130 is then adhered to the harness 106 or leash 110, as needed. Typically, the body component 102 is secured to the harness 106, near the pet's back 606 but can be secured in any suitable position, as is known in the art. Generally, the exterior 608 of the fabric case 130 can be formed from nylon or any other type of weather-resistant and/or water-resistant fabrics.

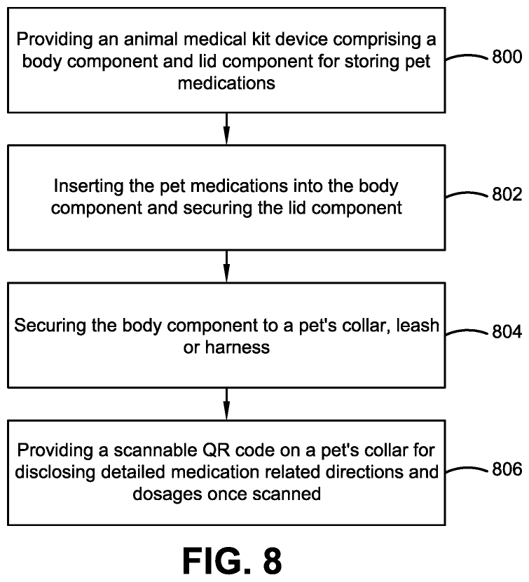

FIG. 8 illustrates a flowchart of the method of ensuring a pet always has access to daily medication. The method includes the steps of at 800, providing an animal medical kit device comprising a body component and lid component for storing pet medications. The method also comprises at 802, inserting the pet medications into the body component and securing the lid component. Further, the method comprises at 804, securing the body component to a pet's collar, leash, or harness. Finally, the method comprises at 806, providing a scannable QR code on a pet's collar for disclosing detailed medication-related directions and dosages once scanned.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “animal medical kit device”, “medical kit device”, “animal device”, and “device” are interchangeable and refer to the animal medical kit device 100 of the present invention.

Notwithstanding the foregoing, the animal medical kit device 100 of the present invention can be of any suitable size and configuration as is known in the art without affecting the overall concept of the invention, provided that it accomplishes the above-stated objectives. One of ordinary skill in the art will appreciate that the animal medical kit device 100 as shown in FIGS. 1-8 is for illustrative purposes only, and that many other sizes and shapes of the animal medical kit device 100 are well within the scope of the present disclosure. Although the dimensions of the animal medical kit device 100 are important design parameters for user convenience, the animal medical kit device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An animal medical kit device that provides a user with a device for ensuring a pet always has access to daily medication, the animal medical kit device comprising:
a body component; and
a lid component;
wherein the body component and the lid component is secured to a pet's harness, collar or leash via a fastener means; and
further wherein the body component is used to store a pet's medication.

2. The animal medical kit device of claim 1, wherein the pet's medication comprises a syringe, nasal spray, an insulin needle, or various pills.

3. The animal medical kit device of claim 2, wherein the body component has two main configurations, one being a cylindrical configuration for retaining a syringe, nasal spray, or an insulin needle, or a second being a rectangular configuration for retaining a variety of pills.

4. The animal medical kit device of claim 3, wherein the cylindrical configuration is a thermos-style, insulated container to keep medications cold, if needed.

5. The animal medical kit device of claim 4, wherein the lid component of the thermos-style, insulated container comprises a twist-on/twist-off, child-proof locking lid.

6. The animal medical kit device of claim 5, wherein the body component of the cylindrical configuration comprises a bottom face, a cylindrical sidewall, extending vertically upwards, and an open top.

7. The animal medical kit device of claim 3, wherein the rectangular configuration comprises a hinged lid.

8. The animal medical kit device of claim 7, wherein the body component of the rectangular configuration comprises a bottom face, an open top, opposing side walls, and opposing front and back walls.

9. The animal medical kit device of claim 3 further comprising a scannable QR code printed on a tag to be secured to a pet's collar.

10. The animal medical kit device of claim 9, wherein the scannable QR code is customized to each pet and includes pet information such as residence and owner phone numbers, as well as medical information, such as detailed medication-related directions and dosages.

11. The animal medical kit device of claim 10, wherein the fastener means for securing the body component of the cylindrical or rectangular configurations to the pet's collar comprises a pair of silicone snapping loops, with one silicone snapping loop secured at each end of the body component.

12. The animal medical kit device of claim 11, wherein the pair of silicone snapping loops each wrap completely around an end of the body component and secure together, then a C-shaped component of each of the silicone snapping loops wrap around a width of the pet's collar with ends snapping into place, preventing the body component from being removed.

13. The animal medical kit device of claim 12, wherein the fastener means for securing the body component of the cylindrical configuration to the pet's harness or leash comprises an adhesive or Velcro-backed fabric case with a lid flap.

14. An animal medical kit device that provides a user with a device for ensuring a pet always has access to daily medication, the animal medical kit device comprising:
a body component with two main configurations, one being a cylindrical configuration for retaining a syringe, nasal spray, or an insulin needle, or a second being a rectangular configuration for retaining a variety of pills;
a lid component; and
a scannable QR code printed on a tag to be secured to a pet's collar;
wherein the body component and the lid component is secured to a pet's harness, collar, or leash via a fastener means;
wherein the body component is used to store a pet's medication;
wherein the pet's medication comprises a syringe, nasal spray, an insulin needle, or various pills;
wherein the scannable QR code is customized to each pet and includes pet information such as residence and owner phone numbers, as well as medical information, such as detailed medication-related directions and dosages;
wherein the fastener means for securing the body component of the cylindrical or rectangular configurations to the pet's collar comprises a pair of silicone snapping loops, with one silicone snapping loop secured at each end of the body component;
wherein the pair of silicone snapping loops each wrap completely around an end of the body component and secure together, then a C-shaped component of each of the silicone snapping loops wrap around a width of the pet's collar with ends snapping into place, preventing the body component from being removed;
wherein the fastener means for securing the body component of the cylindrical configuration to the pet's harness or leash comprises an adhesive or Velcro-backed fabric case with a lid flap; and
further wherein the fastener means for securing the body component of the cylindrical configuration to the pet's leash comprises a keychain or carabiner.

15. The animal medical kit device of claim 14, wherein the body component is attached to the pet's collar at two spaced apart points by the pair of silicone snapping loops, such that the body component will not swing about during a walk.

16. The animal medical kit device of claim 15, wherein if the body component is a rectangular configuration, the pair of silicone snapping loops are secured to a covering with a lid flap, which is sized and shaped to retain the rectangular body component within.

17. The animal medical kit device of claim 14, wherein the adhesive or Velcro-backed fabric case is typically a waterproof, fabric case that comprises a back side of adhesive or Velcro.

18. The animal medical kit device of claim 14 further comprising a plurality of reflective strips applied to an exterior surface of the body component and a plurality of indicia.

19. The animal medical kit device of claim 14 wherein the body component is used to store a human's medicine.

20. A method of ensuring a pet always has access to daily medication, the method comprising the following steps:
providing an animal medical kit device comprising a body component and lid component for storing pet medications;
inserting the pet medications into the body component and securing the lid component; securing the body component to a pet's collar, leash, or harness; and
providing a scannable QR code on a pet's collar for disclosing detailed medication-related directions and dosages once scanned.
